SEQUENCE LISTING (14 sequences)

SEQ ID NO: 1 - protein, 18 aa, Artificial
  218 linker
GSTSGSGKPGSGEGSTKG

SEQ ID NO: 2 - protein, 5 aa, Artificial
  G4S linker
GGGGS

SEQ ID NO: 3 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 4 - protein, 6 aa, Artificial
  Thrombin cleavage sequence
LVPAGS

SEQ ID NO: 5 - DNA, 36 bp, Artificial
  Synthetic primer
ctggtgccacgcggttctttccaggcggccgagcgc

SEQ ID NO: 6 - DNA, 30 bp, Artificial
  Synthetic primer
ggagccaccgccacctaacgtcagacgctg

SEQ ID NO: 7 - DNA, 30 bp, Artificial
  Synthetic primer
ggtggcggtggctccacggacattgtgatg

SEQ ID NO: 8 - DNA, 39 bp, Artificial
  Synthetic primer
gcagatgctaccaagctttcattatgaggagacggtgag

SEQ ID NO: 9 - DNA, 18 bp, Artificial
  Synthetic primer
taatacgactcactatag

SEQ ID NO: 10 - DNA, 30 bp, Artificial
  Synthetic primer
agaaccgcgtggcaccagaccagaagaatg

SEQ ID NO: 11 - protein, 356 aa, Homo sapiens
MAGVACLGKAADADEWCDSGLGSLGPDAAAPGGPGLGAELGPGLSWAPLVFGYVTEDGDTALHLAVIHQHEPFLDFLLGFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGDEYDDIVVHSSRSQTRLPPTPASKPLPDDPRPV

SEQ ID NO: 12 - protein, 306 aa, Homo sapiens
MNGATAAWAPWVRTQRPPEDLGWARSWARGCRGLPSSSATSLRMGTRFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGVSQEERQGSPAGGSG

SEQ ID NO: 13 - protein, 248 aa, Artificial
  Single chain antibody scFvMEL.
TDIVMTQSQKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKGSTSGSGKPGSGEGSTKGEVKVEESGGGLVQPGGSMKLSCVVSGFTFGNYWMNWVRQSPEKGLEWIAEIRLKSNNFARYYAESVKGRFTISRDDSKSSVYLQMINLRAEDTGIYYCTSYGNYVGHYFDHWGQGTTLTVSS

SEQ ID NO: 14 - protein, 571 aa, Artificial
  IkB cell targeting construct IkBalpha/scFvMEL
GSFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTLGGGGSTDIVMTQSQKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKGSTSGSGKPGSGEGSTKGEVKVEESGGGLVQPGGSMKLSCVVSGFTFGNYWMNWVRQSPEKGLEWIAEIRLKSNNFARYYAESVKGRFTISRDDSKSSVYLQMINLRAEDTGIYYCTSYGNYVGHYFDHWGQGTTLTVSS

(19) United States
(12) Patent Application Publication
(10) Pub. No.: US 2007/0202593 A1
(43) Pub. Date: Aug. 30, 2007

(54) Cell-Targeted IKB and Methods for the Use Thereof

(75) Inventors: Yuying Liu, Houston, TX (US); Michael G. Rosenblum, Sugarland, TX (US)

Correspondence Address: FULBRIGHT & JAWORSKI L.L.P., 600 CONGRESS AVE., SUITE 2400, AUSTIN, TX, 78701, US

(73) Assignee: RESEARCH DEVELOPMENT FOUNDATION

(21) Appl. No.: 11/679,630

(22) Filed: Feb. 27, 2007

Related U.S. Application Data
(60) Provisional application No. 60/777,016, filed on Feb. 27, 2006.

(51) Int. Cl. C12N 7/00; C12N 5/00; C12N 7/01; C12N 5/02

(52) U.S. Cl. 435/325; 435/235.1

(57) ABSTRACT

Activation of nuclear factor kB (NF-kB) is involved in a number of diseases such as viral and bacterial infections, and cell proliferative disorders such as cancer and autoimmune disease. In certain instances, constitutive NF-kB activity has also been liked to the resistance of certain cancers to chemo and radiation therapy. The instant invention concerns method of inhibiting NF-kB activity in target cell populations by deliver of a polypeptide inhibitor of NF-kB (IkB). Methods of the invention may be used to treat diseases such as infections, and cell proliferative disorders. Methods for sensitizing cells to apoptosis and cytotoxic therapies are also described.

This application claims the benefit of U.S. Provisional Patent Application No. 60/777,016, filed on Feb. 27, 2006.

### BACKGROUND OF THE INVENTION

1. Field of the Invention

The instant invention concerns cell targeted therapeutic compositions and method for their uses. NF-kB activity may be specifically down-regulated in cells by directed delivery compositions such as the protein inhibitor of NF-kB, IkB.

2. Description of Related Art

Nuclear Factor κB (NF-kB) is transcription factor that plays a crucial role in cell proliferation, cancer, apoptosis and inflammatory responses. Five members of the NF-kB family have been identified in mammals: p50 (NF-kB-1), p52 (NF-kB-2), p65 (Rel A), c-Rel, and RelB. These proteins are present in cells as homo- or heterodimers; however, the most common transcription-competent form is the p50/p65 dimer. All members share a Rel homology domain, which mediates dimerization, nuclear translocation, DNA binding, and interaction with the IkB family of proteins (Baldwin, 1996; Ghosh et al., 1998). Regulation of NF-kB is mainly controlled by the inhibitory IkB proteins, which include IkBα. IkB proteins interact with NF-kB via their ankyrin repeats and retain the transcription factor in the cytoplasm in non-stimulated cells. After cell stimulation, IkB is rapidly phosphorylated in an N-terminal recognition motif by the IkB kinase (IKK) 3 complex, which comprises two kinases, IKKα (IKK-1) and IKKβ (IKK-2), and a third molecule, IKKγ or NF-kB essential modulator (NEMO) (Agou et al., 2004; Karin 1999). Phosphorylated IkBs become polyubiquitinated and are subsequently degraded by the 26S proteasome. The best-characterized member is IkBα, which is posphorylated on serines 32 and 36 by the IKK complex (Traenckner et al., 1995). Degradation of IkB exposes a nuclear localization signal on NF-kB, which mediates its translocation to the nucleus to initiate gene transcription. Dominant-negative mutants IkBα (IkBαM) have been engineered, which cannot be phosphorylated and degraded; thus, NF-kB activity is constitutively repressed in cells transfected with IkBαM. The stable expression of IkBαM has been shown to inhibit the activation of NF-kB in a variety of cell types (Pajonk et al., 1999).

Constitutive activation of NF-kB plays a role in a variety of human malignancies such as pancreatic cancer, colon cancer, breast cancer, T-cell leukemias, and lymphomas. Several reports have also demonstrated that NF-kB is constitutively activated in human melanoma cells (Huang et al., 2000; Yang and Richmond, 2001), and it has recently been shown that this constitutive activity is a result of elevated IkB kinase (IKK) activity arising from aberrant NF-kB-inducing kinase activation (Dhawan and Richmond, 2002). Recent studies have shown NF-kB to be a critical regulator of apoptosis by controlling the transcription of genes with products that block cell death (Aggarwal et al., 2004; Aggarwal et al., 2005). For example constitutive activation of NF-kB induces overexpression of downstream targets such as Bcl-XL, Bcl-2, vascular endothelial growth factor (VEGF), and interleukin-8, which may in turn mediate resistance to apoptosis and contribute to the resistance of some cancers to chemotherapy and radiation. Several genes involved in tumor initiation, promotion, and metastasis are also regulated by NF-kB, suggesting that NF-kB acts as a mediator of tumor genesis and can thus be used as target for chemoprevention and treatment of cancer. Agents that suppress NF-kB activation have been shown to suppress the expression of genes involved in carcinogenesis and tumor genesis in vivo (Barkett et al., 1999).

Given the central role of NF-kB in immune response, autoimmune disease and cancer, many anti-inflammatory and chemotherapeutic agents are targeted to the NF-kB pathway (U.S. Pat. No. 7,083,957 and U.S. Pat. No. 5,891,924). One possible approach to NF-kB inhibition is use of the endogenous NF-kB inhibitor molecule known as IκB as a therapeutic. The IkB polypeptide is able to specifically bind to NF-kB and sequester it in an inactive form in the cytoplasm of cells. Thus, IkB administration enables specific down regulation of NF-kB function via the natural cellular pathway. Previously it has been shown that IkB can be delivered to cells in nucleic acid form via an adenoviral vector (Batra et al., 1999; Iimuro et al., 1998; Foxwell et al., 2000). However, these techniques have certain disadvantages since IkB can only be expressed in cells that are susceptible to adenovirus infection, and it is not possible to target IkB deliver to any particular type of cell with in this population. Other approaches for delivery of IkB have similar limitations. For instance, IkB can be fused with a membrane translocating peptide, but there is no way of targeting the IkB fusions to cell populations of interest, thus limiting the therapeutic usefulness of these molecules (WO 2005/017188). Despite the potential effectiveness of direct IkB inhibition of the NF-kB pathway, previously there have been no effective methods for targeted delivery of IkB to cell populations of interest.

### SUMMARY OF THE INVENTION

The instant invention provides a significant improvement over the prior art methods of inhibiting NF-kB by enabling cell targeted delivery of IkB. Delivery to specific cell types enables improved methods of treating diseases such as bacterial and viral infections as well as cell proliferative diseases, such as cancer and autoimmune disease. In particular, compositions and methods of the invention enable cell targeted enhancement of apoptosis and thus can enhance the ability of known therapeutic agents to kill a targeted cell population.

In one embodiment of the invention there is provided a cell targeting construct comprising a polypeptide inhibitor of NF-kB (IkB) conjugated to a cell targeting moiety. As used herein the term “polypeptide inhibitor of NF-kB” (IkB) means a polypeptide that is able to bind to the Rel homology domain of one or more NF-kB family member(s) thereby reducing NF-kB activity. For example, IkB molecules for use in the current invention include but are not limited to human IkBα (SEQ ID NO:3), human IkBβ isoform a (SEQ ID NO:11), human IkB isoform b (SEQ ID NO:12) or any derivative of the foregoing. IkB polypeptides for use according to the invention are further detailed below.

In some aspects of the invention the polypeptide inhibitor of NF-kB and cell targeting moiety may be chemically conjugated, either covalently or non-covalently. For example, a covalent chemical conjugate may be conjugated by SMPT cross-linking. In certain instances, an IkB and cell targeting moiety may be conjugated via a non-covalent interaction, such as by biotin-avidin conjugation (e.g., see U.S. Pat. No. 6,214,974). In these applications the IkB and cell targeting moiety are generated separately and subsequently conjugated to one another. However, in a preferred embodiment, the polypeptide inhibitor of NF-kB and cell targeting moiety are comprised in a fusion protein. These types of conjugates have a number of advantages for example simplified synthesis and purification.

In some specific embodiments of the invention, there is provided a cell targeting construct comprising IkB conjugated to a cell targeting moiety wherein the cell targeting construct is a fusion protein. Furthermore, in certain aspects of the invention, there is provided a nucleic acid that encodes a cell targeting construct according to the invention. Nucleic acids according to the invention preferably comprise additional sequences such as sequences to facilitate the expression of a cell targeting construct in a eukaryotic or a prokaryotic cell.

In the case where the cell targeting construct is a fusion protein it is envisioned that the IkB may be positioned either amino terminal (NH₂) or carboxy terminal (COO⁻) with respect to the cell targeting moiety. Thus, while in certain embodiments a fusion protein according to the invention may comprise NH₂—X-IkB-X-cell targeting moiety-X—COO⁻, in other cases the fusion protein may be arranged in the opposite orientation; NH₂—X-cell targeting moiety-X-IkB-X—COO⁻. In each case X indicates a position where additional amino acids may be inserted. Thus, in either orientation the cell targeting construct may comprise additional amino acids at the amino terminus, the carboxy terminus or between the IkB and the cell targeting moiety.

It will be understood that in certain cases, a fusion protein may comprise additional amino acids positioned between the IkB and the cell targeting polypeptide. In general these sequence are termed “linker sequences” or “linker regions.” One of skill in the art will recognize that linker regions may be one or more amino acids in length and are often comprise one or more glycine residues which confer flexibility to the linker. In some specific examples, linkers for use in the current invention include the 218 linker (GSTSGSGKPGSGQGSTKG) (SEQ ID NO:1) and the G₄S linker (GGGGS) (SEQ ID NO:2). For instance, in some applications, a linker region may comprise a protease cleavage site, such as the cleavage site recognized by an endogenous intracellular protease. In this case when the cell targeting construct is internalized into a target cell proteolytic cleavage will separate the IkB polypeptide from the cell targeting moiety. Cell targeting constructs according to this embodiment may have the advantage enhanced intracellular activity of the targeted IkB since potential interference from the cell targeting polypeptide will be reduced.

Cell targeting constructs according to the invention may comprise additional amino acids attached to IkB, the cell targeting moiety, or both. For example, additional amino acids may be included to aid production or purification of a cell targeting construct. Some specific examples of amino acid sequences that may be attached to cell targeting moiety include, but are not limited to, purification tags, proteolytic cleavage sites, such as a Thrombin cleavage site (SEQ ID NO:4), intracellular localization signals or secretion signals.

A cell targeting construct according to the invention will desirably have two properties; (1) binding affinity for a specific population cells and (2) the ability to be internalized into a specific population of cells. It is envisioned, however, that even cell targeting constructs that are poorly internalized by be used in methods according to the instant invention. Methods well known to those in the art may be used to determine whether a particular cell targeting construct is internalized by target cells, for example by immunohistochemical staining or immunoblot of intracellular extracts may be employed both of which are exemplified herein. It is also envisioned that in certain cases cell targeting moieties that can not, by themselves be internalized, may be internalized in the context of the cell targeting constructs according to the invention. Cell targeting moieties for use in the invention include but are not limited to antibodies, growth factors, hormones, peptides, aptamers, avimers (see for example U.S. Patent Applns. 20060234299 and 20060223114) and cytokines. As discussed above cell targeting moieties may be conjugated to IkB via a covalent or non-covalent linkage, and in certain cases the targeting construct may be a fusion protein.

In certain preferred embodiments, cell targeting moieties for use in the current invention are antibodies. In general the term antibody includes, but is not limited to, polyclonal antibodies, monoclonal antibodies, single chain antibodies, humanized antibodies, minibodies, dibodies, tribodies as well as antibody fragments, such as Fab′, Fab, F(ab′)2, single domain antibodies and any mixture thereof. In some cases it is preferred that the cell targeting moiety is a single chain antibody (scFv). In a related embodiment, the cell targeting domain may be an avimer polypeptide. Therefore, in certain cases the cell targeting constructs of the invention are fusion proteins comprising IkB and a scFv or an avimer. For example, in some very specific embodiments the cell targeting construct is a fusion protein comprising IkB fused to scFvMEL (SEQ ID NO:13) or to scFv23.

In certain aspects of the invention, a cell targeting moieties may be a growth factor. For example, transforming growth factor, epidermal growth factor, insulin-like growth factor, fibroblast growth factor, B lymphocyte stimulator (BLyS), heregulin, platelet-derived growth factor, vascular endothelial growth factor (VEGF), or hypoxia inducible factor may be used as a cell targeting moiety according to the invention. These growth factors enable the targeting of constructs to cells that express the cognate growth factor receptors. For example, VEGF can be used to target cells that express FLK-1 and/or Flt-1. In still further aspects the cell targeting moiety may be a polypeptide BLyS (see U.S. Patent Appln. 20060171919).

In further aspects of the invention, a cell targeting moiety may be a hormone. Some examples of hormones for use in the invention include, but are not limited to, human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY3-36, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36. As discussed above targeting constructs that comprise a hormone enable method of targeting cell populations that comprise extracellular receptors for the indicated hormone.

In yet further embodiments of the invention, cell targeting moieties may be cytokines. For example, IL1, IL2, IL3, IL4, IL5, IL6, IL7, IL8, IL9, IL10, IL11, IL12, IL13, IL14, IL15, IL-16, IL-17, IL-18, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, IFN-γ, IFN-α, IFN-β, LT-β, CD40 ligand, Fas ligand, CD27 ligand, CD30 ligand, 4-1BBL, TGF-β, IL 1α, IL-1β, IL-1 RA, MIF and IGIF may all be used as targeting moieties according to the invention.

From the foregoing description it will be clear to one of skill in the art that cell targeting constructs according to the invention may target particular populations of cells depending on the cell targeting moiety that is employed. For instance, the cell targeting moiety may be an infected cell targeting moiety. In this case the cell targeting moiety may bind to cellular protein that primarily expressed on the surface of cells that are infected by a pathogen such as bacteria, a protozoan or a virus. In certain other aspects, the cell targeting moiety may bind to a factor encoded by the pathogen such as a bacterial, protozoal or viral protein. In this aspect it is envisioned that cell targeting constructs may be indirectly targeted to cells by binding to a pathogen before or as it enters a target cell. Thus, the transit of a pathogen into a cell may, in some instances, mediate internalization of the targeting construct. In additional aspects, cell targeting moieties may bind to polypeptides encoded by the pathogen that are expressed on the surface of infected cells. For example, in the case of cell infected with human immunodeficiency virus (HIV) a cell targeting moiety may bind to, for example, gp120. It is envisioned that any of the foregoing methods may be used to limit the spread of infection. For example, delivery of IkB to the infected cell may induce apoptosis or sensitize a cell to undergo apoptosis.

In some aspects of the invention it is contemplated that a cell targeting moiety for use in the current invention may be defined as an immune cell targeting moiety. In this case the cell targeting moiety may bind to and be internalized by a cell surface molecule that is expressed on a specific populations of immune cells. Targeting IkB to certain types of immune cells may be used, for example, to treat autoimmune diseases.

In yet further aspects of the invention a cell targeting moiety of the invention may be a cancer cell targeting moiety. It is well known that certain types of cancer cells aberrantly express surface molecules that are unique as compared to surrounding tissue. Thus, cell targeting moieties that bind to these surface molecules enable the targeted delivery of IkB specifically to the cancers cells. For example, a cell targeting moiety may bind to and be internalized by a lung, breast, brain, prostate, spleen, pancreatic, cervical, ovarian, head and neck, esophageal, liver, skin, kidney, leukemia, bone, testicular, colon or bladder cancer cell. The skilled artsan will understand that the effectiveness of cancer cell targeted IkB may, in some cases, be contingent upon the expression or exprerssion level of a particular cancer marker on the cancer cell. Thus, in ceratin aspects there is provided a method for treating a cancer with targeted IkB comprising determining whether (or to what extent) the cancer cell expresses a particular cell surface marker and adminsierting IkB targeted therapy (or another anticancer therapy) to the cancer cells depending on the expression level of a moarker gene or polypeptide.

Thus, in certain embodiments of the invention, there is provided a method for treating a cell proliferative disease comprising administering a cell targeting construct according to the invention. As used herein the phrase “cell proliferative condition” includes but is not limited to autoimmune diseases, cancers and precancerous conditions. For example, methods of the invention may used for the treatment of cancers such as lung, breast, brain, prostate, spleen, pancreatic, cervical, ovarian, head and neck, esophageal, liver, skin, kidney, leukemia, bone, testicular, colon, or bladder cancer. In a very specific example, there is provided a method for treating a skin cancer such as a melanoma. For instance, there is provided a method for treating a gp240 positive skin cancer comprising, for example administering IkB/scFvMEL.

In some cases, cell targeting constructs of the invention are used in combination with cytotoxic therapies. Thus, in certain instances, there is provided a method sensitizing cells to a cytotoxic therapy by administering a cell targeting construct comprising an IkB conjugated to a cell targeting moiety. In this case the cell targeting construct may be administered prior to, concurrently with, or after administration of the cytotoxic therapy. For example, a cytotoxic therapy may be chemotherapy, radiation therapy, gene therapy or immunotherapy. If the combined cytotoxic therapy is a chemotherapy in may be preferred that the chemotherapy comprise one or more additional NF-kB inhibitors. Some examples of NF-kB inhibitors for use in methods of the invention include but are not limited to curcuminoids, avicins (see, for example, U.S. Pat. No. 6,444,233), CAPE, capsaicin, sanguinarin, a PTPase inhibitor, lapachone, resveratrol, vesnarinone, leflunomide, anethole, a PI3 kinase inhibitor, oleanderin, emodin, a serine preotease inhibitor, a protein tyrosine kinase inhibitor, thalidomide, methotrexate or a combination or derivative thereof. In certain additional embodiments the chemotherapy may comprise administration of paclitaxel, gemcitabin, 5-fluorouracil, etoposide, cisplatin, capothecin, vincristine, Velcade, doxorubicin or a combination or derivative thereof.

In yet further aspects of the invention there is provided a method for treating an autoimmune disease or an inflammatory disease comprising administering a cell targeting construct according the invention. For example, cell targeting molecules according to the invention may be used in the treatment of rheumatoid arthritis, psoriasis, osteoarthritis, inflammatory bowel disease, type 1 diabetes, tissue or organ rejection or multiple sclerosis. In these aspects of the invention cell targeting constructs may be used in combination with other treatment regimens, such as steroids. In these applications cell targeting constructs of the invention offer several advantages over currently available treatments. For example, by targeting specific cell populations autoimmunity and/or inflammation my be reduced with-out the general immunosuppressive effects that are exhibited by many current therapies.

Embodiments discussed in the context of a methods and/or composition of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIG. 1: An example schematic representation of a gene encoding IκBα/scFvMEL. The amino acid sequence of this particular cell targeting construct (after thrombin cleavage) is indicated in SEQ ID NO:14.

FIG. 2: Internalization of IκBα/scFvMEL into gp240 antigen positive human melanoma cells in culture. Cell lines A375-M (gp240+), AAB527 (gp240+) and TXM-1 (gp240(−)) were treated with IκBα/scFvMEL for 2 hours at the indicated concentrations (nM). Cells were lysed and proteins were analyzed by Western blot. The migration of ectopic IκBα/scFvMEL and of endogenous IkBα shown with arrows. Western blot analysis of intracellular actin was used to demonstrate equal loading in each case.

FIG. 3: IkBα/scFvMEL fusion construct is localized to tumor tissues in vivo. Mice bearing A375-M xenograft tumors were administered intravenously IkBα/scFvMEL (100 mg/kg). Twenty-four hours after the last dose, animals were sacrificed, and tumor tissues were removed, fixed, and subjected to immunohistochemical staining for IkBα/scFvMEL (anti-scFvMEL antibody). Localization and internalization of IkBα/scFvMEL was observed in tumor tissues in the treatment group, but not in the control group.

FIG. 4A-C: FIG. 4A, IkBα/scFvMEL fusion construct blocks constitutive and radiation-induced NF-kB activity in melanoma cells. Gp240 antigen positive A375-M and A375SM cells as well as gp240 antigen negative TXM-1 cells were exposed to 4 Gy and/or treated with 0.3 μM IkBα/scFvMEL for 2 hours as indicated. Cells were harvested 2 hours posttreatment and the amount of active NF-kB was determined by EMSA. FIG. 4B-C, treatment with IkBα/scFvMEL sensitizes gp240 antigen positive melanoma cells to ionizing radiation. Radiosensitization by IkBα/scFvMEL was based on clonogenic cell survival assays. A375-M (FIG. 4B) and TXM-1 (FIG. 4C) cells were pre-treated with IkBα/scFvMEL (0.3 μM for 2 hours), the drug washed off, and cells were irradiated at various doses and plated for clonogenic cell survival assay. Observed sensitizations were statistically significant in both the 2 and 4 Gy dosage groups on A375-M cells (p<0.05). No statistically significant sensitization was observed in gp240 antigen negative TXM-1 cells (p>0.05).

FIG. 5A-B: Decrease in levels of Bcl-2 and Bcl-XL in cultured A375-M cells and A375-M xenograft tumors following treatment with IkBα/scFvMEL. Western blot analysis for Bcl-2 and Bcl-XL in A375-M and TXM-1 cells treated with 0.2 μM for 2 hours (FIG. 5A) or A375-M xenograft tumors (FIG. 5B). Mice bearing A375-M xenograft tumors were administered intravenously IkBα/scFvMEL (100 mg/kg). Twenty-four hours after the last dose, animals were sacrificed, and tumor tissues were removed and homogenized in ice-cold lysis buffer with protease inhibitors. Protein concentration in each supernatant was determined and equal amounts of protein were analyzed.

FIG. 6: In vivo antitumor activity of IkB/scFvMEL on A375 xenograft tumors. Mice comprising A375 tumor xenografts were injected with IkB/scFvMEL (solid triangles) or vehical control (solid squares) at the indicated times (see arrows in the x-axis) and total tumor volume was monitored.

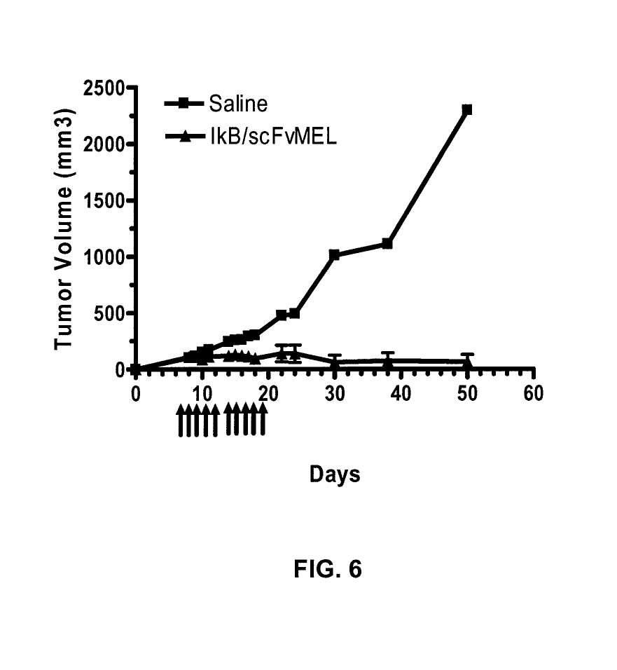

### DETAILED DESCRIPTION OF THE INVENTION

Nuclear factor kB (NF-kB) is a transcription factor that is involved in a variety of disease conditions. A number of small molecule NF-kB inhibitors are currently in use or under development for the treatment of cancer, autoimmune disease and infectious disease (viral infection). However, current inhibitors are limited in their effectivness since they can not be targeted to particular cell populations and may have activity that is not specific to the NF-kB pathway. Both of these limitations of previous NF-kB inhibitors may contribute to undesirable side effects and both limitations are addressed by the compositions and methods presented herein.

Compositions and methods described herein concern cell targeted deliver of IkB, a polypeptide that is acts as highly specific NF-kB inhibitor. One application for such compositions and methods is the treatment of cell proliferative diseases such as cancer. In particular, cell targeting constructs may be used to sensitize cells to cytotoxic therapies such as chemotherapy and/or radiation therapy. Thus, the instant invention provides, in certain embodiments, a novel treatment for cancers that have acquired resistance to cytotoxic agents or therapies. According to the methods described herein cancers that are resistant to cytotoxic agents may be sensitized or resensitized to a cytotoxic therapy by administration of cell targeted IkB, as described herein.

Another cell proliferative disease in which NF-kB plays a role is autoimmune disease. In this case NF-kB activity can prevent immune cells from undergoing apoptosis, in some cases resulting in aberrant proliferation and elevated inflammatory response. Thus, NF-kB inhibitors can be used to control autoimmune disease by sensitizing or resensitizing immune cells to apoptotic signals. Since methods of the current invention enable the delivery of IkB specifically to immune cells of interest it is envisioned that this targeted therapy will enable more effective and these detrimental method for treating autoimmune diseases.

Additional aspects and methods for the construction and use of targeted IkB molecules are discussed below.

I. IκB Molecules

As described in the foregoing summary, some aspects the invention concern a cell targeting construct that comprises a polypeptide inhibitor of NF-kB (IkB) and a cell targeting moeity. IkB molecules for use in the current invention include, but are not limited to, human IkBα (SEQ ID NO:3), human IkBβ isoform a (SEQ ID NO:11), human IkBβ isoform b (SEQ ID NO:12) or a derivative of the foregoing. For instance an IkB sequence for use according to the current invention may comprise an IkB that at least 70%, 80%, 90%, 95%, 98% or more identical to human IkBα and/or either of the two human IkBβ protein isoforms. Thus, in certain aspects of the invention an IkB may be a human IkBα sequence wherein one or more amino acid has been substituted for an amino acid at a corresponding position of a human IkBβ isoform.

In certain additional cases, IkB for use in the targeting constructs of the invention may be an IkB from a non-human source. For example an IkB may be a murine IkBα (NCBI accession No. AAA79696), a murine IkBβ (NCBI accession No. NP_{—}035038), a rat IkBα (NCBI accession No. XP_{—}343066), a rat IkBβ (NCBI accession No. NP_{—}110494), a pig IkBα (NCBI accession No. CAA84619), each incorporated herein by reference, or any other mammalian IkB polypeptide. Thus, in certain aspects of the invention an IkB may be a human IkB sequence wherein one or more amino acids has been substituted for an amino acid at the corresponding position of an IkB protein from a different species. For example, an amino acid from human IkBα may be substituted for an amino acid at the corresponding position of murine IkBα (NCBI accession No. AAA79696), murine IkBβ (NCBI accession No. NP_{—}035038) rat IkBα (NCBI accession No. XP_{—}343066), rat IkBβ (NCBI accession No. NP_{—}110494), pig IkBα (NCBI accession No. CAA84619) or any other mammalian IkB polypeptide.

In additional aspects of the invention, IkB polypeptides may be further modified by one or more amino substitutions while maintaining their ability to inhibit NF-kB activity. For example, amino acid substitutions can be made at one or more positions wherein the substitution is for an amino acid having a similar hydrophilicity. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Thus such conservative substitution can be made in IkB and will likely only have minor effects on their activity and ability to repress NF-kB activity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5); leucine (−1.8); isoleucine (−1.8); tyrosine (2.3); phenylalanine (−2.5); tryptophan (−3.4). These values can be used as a guide and thus substitution of amino acids whose hydrophilicity values are within ±2 are preferred, those that are within ±1 are particularly preferred, and those within ±0.5 are even more particularly preferred. Thus, any of the IkB polypeptides described herein may be modified by the substitution of an amino acid, for different, but homologous amino acid with a similar hydrophilicity value. Amino acids with hydrophilicities within +/−1.0, or +/−0.5 points are considered homologous.

Furthermore, it is envisioned that IkB sequences may be modified by amino acid deletions, substitutions, additions or insertions in order to enhance the ability of the polypeptide to repress NF-kB in targeted cells. For example, mutant IkB proteins that can not be phosphorylated in the cell have been shown to mediate enhanced NF-kB repression. An example is IkBαM, a modified human IkBα wherein the serines at positions 32 and 36 have been mutated to alanine (Pajonk et al., 1999, incorporated herein by reference). These changes prevent phosphorylation dependent degradation of the protein and enhance repression of NF-kB. It will be understood by one of skill in the art that similar mutations can be introduced in any IkB protein or derivative so as to mutate residues that are phosphorylated by an IkB kinase. Additionally, IkB super repressors have been described that have the ability to bind to wider range of NF-kB subunits. These proteins may have an enhanced ability to repress NF-kB activity (WO 2005/021722, incorporated herein by reference). Any of the enhanced IkB polypeptides may also be used in the compositions and methods of the current invention.

II. Cell Targeting Moieties

As discussed above cell targeting moieties according to the invention may be, for example, an antibody, a growth factor, a hormone, a peptide, an aptamer or a cytokine. For instance, a cell targeting moiety according the invention may bind to a skin cancer cell such as a melanoma cell. It has been demonstrated that the gp240 antigen is expressed in variety of melanomas but not in normal tissues. Thus, in certain aspects of the invention, there is provided a cell targeting construct comprising an IkB and a cell targeting moiety that binds to gp240. In some instances, the gp240 binding molecule may be an antibody, such as the ZME-018 (225.28S) antibody or the 9.2.27 antibody. In an even more preferred embodiment, the gp240 binding molecule may be a single chain antibody such as the scFvMEL antibody. Therefore, in a very specific embodiment of the invention, there is provided a cell targeting construct comprising human IkBα conjugated to scFvMEL.

In yet further specific embodiments of the invention, cell targeting constructs may be directed to breast cancer cells. For example cell targeting moieties that bind to Her-2/neu, such as anti-Her-2/neu antibodies may conjugated to IkB. One example of a such a cell targeting constructs are fusion proteins comprising the single chain anti-Her-2/neu antibody scFv23 and IkB. Other scFv antibodies such as scFv(FRP5) that bind to Her-2/neu may also be used in the compositions and methods of the current invention (von Minckwitz et al., 2005).

In certain additional embodiments of the invention, it is envisioned that cancer cell targeting moieties according to invention may have the ability to bind to multiple types of cancer cells. For example, the 8H9 monoclonal antibody and the single chain antibodies derived therefrom bind to a glycoprotein that is expressed on breast cancers, sarcomas and neuroblastomas (Onda et al., 2004). Another example are the cell targeting agents described in U.S. patent application no. 2004005647 and in Winthrop et al., 2003 that bind to MUC-1 an antigen that is expressed on a variety cancer types. Thus, it will be understood that in certain embodiments, cell targeting constructs according the invention may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy.

Since a large number of cell surface receptors have been identified in hematopoietic cells of various lineages, ligands or antibodies specific for these receptors may be used as cell-specific targeting moieties. IL2 may also be used as a cell-specific targeting moiety in a chimeric protein to target IL2R+ cells. Alternatively, other molecules such as B7-1, B7-2 and CD40 may be used to specifically target activated T cells (The Leucocyte Antigen Facts Book, 1993, Barclay et al. (eds.), Academic Press). Furthermore, B cells express CD19, CD40 and IL4 receptor and may be targeted by moieties that bind these receptors, such as CD40 ligand, IL4, IL5, IL6 and CD28. The elimination of immune cells such as T cells and B cells is particularly useful in the treatment of autoimmunity, hypersensitivity, transplantation rejection responses and in the treatment of lymphoid tumors. Examples of autoimmune diseases are multiple sclerosis, rheumatoid arthritis, insulin-dependent diabetes mellitus, systemic lupus erythemotisis, scleroderma, and uviatis. More specifically, since myelin basic protein is known to be the major target of immune cell attack in multiple sclerosis, this protein may be used as a cell-specific targeting moiety for the treatment of multiple sclerosis (WO 97/19179; Becker et al., 1997).

Other cytokines that may be used to target specific cell subsets include the interleukins (IL1 through IL15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego).

A skilled artisan recognizes that there are a variety of known cytokines, including hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-1BBL)); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)). Furthermore, the Fc portion of the heavy chain of an antibody may be used to target Fc receptor-expressing cells such as the use of the Fc portion of an IgE antibody to target mast cells and basophils.

Over the past few years, several monoclonal antibodies have been approved for therapeutic use and have achieved significant clinical and commercial success. Much of the clinical utility of monoclonal antibodies results from the affinity and specificity with which they bind to their targets, as well as long circulating life due to their relatively large size. Monoclonal antibodies, however, are not well suited for use in indications where a short half-life is advantageous or where their large size inhibits them physically from reaching the area of potential therapeutic activity.

Thus, in a highly preferred embodiments, cell targeting moieties according to the invention are antibodies or avimers. Antibodies and avimers can be generated to virtually any cell surface marker thus, providing a method for targeted to delivery of IkB to virtually any cell population of interest. Methods for generating antibodies that may be used as cell targeting moieties are detailed below. Methods for generating avimers that bind to a given cell surface marker are detailed in U.S. Patent Applns. 20060234299 and 20060223114, each incorporated herein by reference.

III. Methods for Producing Antibodies

As described above certain aspects of the invention involve to use of antibodies as cell targeting moieties. Antibodies may be made by any of the methods that as well known to those of skill in the art. The following methods exemplify some of the most common antibody production methods.

1. Polyclonal Antibodies

Polyclonal antibodies generally are raised in animals by multiple subcutaneous (sc) or intraperitoneal (ip) injections of the antigen. As used herein the term “antigen” refers to any polypeptide that will be used in the production of a antibodies. However it will be understood by one of skill in the art that in many cases antigens comprise more material that merely a single polypeptide. For example in certain aspects of the invention it is preferred that antibodies recognize cancer cells, thus in certain aspects of the invention an antigen may comprise one or more tumor cells. In certain other aspects of the invention antibodies will be generated against specific polypeptide antigens. For example antibodies can be made against polypeptides that have been identified to be expressed on the surface of cancer cells, such as gp240, MUC-1 or Her-2/neu. Thus one of skill it the art would easily be able to generate an antibody that binds to any particular cell or polypeptide of interest using method that are well known in the art.

In the case where an antibody is to be generated that binds to a particular polypeptide it may be useful to conjugate the antigen or a fragment containing the target amino acid sequence to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹N═C═NR, where R and R¹ are different alkyl groups.

Animals are immunized against the immunogenic conjugates or derivatives by, for example, combining 1 mg or 1 μg of conjugate (for rabbits or mice, respectively) with 3 volumes of Freud's complete adjuvant and injecting the solution intradermally at multiple sites. One month later the animals are boosted with about ⅕ to 1/10 the original amount of conjugate in Freud's complete adjuvant by subcutaneous injection at multiple sites. 7 to 14 days later the animals are bled and the serum is assayed for specific antibody titer. Animals are boosted until the titer plateaus. Preferably, the animal is boosted with the same antigen conjugate, but conjugated to a different protein and/or through a different cross-linking reagent. Conjugates also can be made in recombinant cell culture as protein fusions. Also, aggregating agents such as alum are used to enhance the immune response.

2. Monoclonal Antibodies

In preferred embodiments of the invention the cell targeting moiety is a monoclonal antibody. By using monoclonal antibodies cell targeting constructs of the invention can have greater specificity for a target cell than targeting moieties that employ polyclonal antibodies. Monoclonal antibodies are obtained from a population of substantially homogeneous antibodies, i.e. the individual antibodies comprising the population are identical except for possible naturally-occurring mutations that may be present in minor amounts. Thus, the modifier “monoclonal” indicates the character of the antibody as not being a mixture of discrete antibodies.

For example, monoclonal antibodies of the invention may be made using the hybridoma method first described by Kohler & Milstein (1975), or may be made by recombinant DNA methods (Cabilly et al.; U.S. Pat. No. 4,816,567).

In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding 1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the target antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson & Pollard (1980).

After hybridoma cells are identified that produce antibodies of the desired specificity, affinity, and/or activity, the clones may be subcloned by limiting dilution procedures and grown by standard methods, Goding (1986). Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies of the invention may be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison et al. (1984), or by covalently joining to the immunoglobulin coding sequence all or part of the coding sequence for a non-immunoglobulin polypeptide. In that manner, “chimeric” or “hybrid” antibodies are prepared that have the binding specificity for any particular antigen described herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for the target antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

For diagnostic applications, the antibodies of the invention typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; biotin; radioactive isotopic labels, such as, e.g., ³H, ¹⁴C, ³²P, ³⁵S, or ¹²⁵I, or an enzyme (i.e., either by chemical coupling or by generating a fusion protein), such as alkaline phosphatase, beta-galactosidase or horseradish peroxidase.

Any method known in the art for separately conjugating the antibody to the detectable moiety may be employed, including those methods described by Hunter et al. (1962); David et al. (1974); Pain et al. (1981); and Nygren (1982).

The antibodies of the present invention may be employed in any known assay method, such as competitive binding assays, direct and indirect sandwich assays, and immunoprecipitation assays (Zola, 1987).

Competitive binding assays rely on the ability of a labeled standard (which may be a purified target antigen or an immunologically reactive portion thereof) to compete with the test sample analyte for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. David & Greene, U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

3. Humanized Antibodies

As discussed previously, antibodies for use in the methods of the invention may be polyclonal or monoclonal antibodies or fragments thereof. However, in some aspects it is preferred that the antibodies are humanized such that they do not elicit an immune response in subject being treated. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., 1986); Riechmann et al., 1988; Verhoeyen et al., 1988), by substituting rodent CDRs or CDR sequences for the corresponding sequences of a human antibody. Accordingly, such “humanized” antibodies are chimeric antibodies (Cabilly), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties, for example the ability to be internalized into cells. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. For further details see U.S. Pat. No. 5,821,337.

4. Human Antibodies

Human monoclonal antibodies can be made by the hybridoma method. Human myeloma and mouse-human heteromyeloma cell lines for the production of human monoclonal antibodies have been described, for example, by Kozbor (1984) and Brodeur et al. (1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al. (1993); Jakobovits et al. (1993).

Alternatively, the phage display technology (McCafferty et al., 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle.

Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; see Winthrop et al., 2003 or for a review see, e.g. Johnson et al. (1993). Several sources of V-gene segments can be used for phage display. Clackson et al. (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al. (1991), or Griffith et al. (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This techniques allows the production of antibodies and antibody fragments with affinities in the nM range. A strategy for making very large phage antibody repertoires (also known as “the mother-of-all libraries”) has been described by Waterhouse et al. (1993), and the isolation of a high affinity human antibody directly from such large phage library has been reported. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213). Unlike traditional humanization of rodent antibodies by CDR grafting, this technique provides completely human antibodies, which have no framework or CDR residues of rodent origin.

5. Single Chain Antibodies

Single chain antibodies (SCAs) are genetically engineered proteins designed to expand on the therapeutic and diagnostic applications possible with monoclonal antibodies. SCAs have the binding specificity and affinity of monoclonal antibodies and, in their native form, are about one-fifth to one-sixth of the size of a monoclonal antibody, typically giving them very short half-lives. Human SCAs offer many benefits compared to most monoclonal antibodies, including more specific localization to target sites in the body, faster clearance from the body, and a better opportunity to be used orally, intranasally, transdermally or by inhalation, for example. In addition to these benefits, fully-human SCAs can be isolated directly from human SCA libraries without the need for costly and time consuming “humanization” procedures. SCAs are also readily produced through intracellular expression (inside cells) allowing for their use in gene therapy applications where SCA molecules act as specific inhibitors of cell function.

Single-chain recombinant antibodies (scFvs) consist of the antibody VL and VH domains linked by a designed flexible peptide tether (Atwell et al., 1999). Compared to intact IgGs, scFvs have the advantages of smaller size and structural simplicity with comparable antigen-binding affinities, and they can be more stable than the analogous 2-chain Fab fragments (Colcher et al., 1998; Adams and Schier, 1999). Several studies have shown that the smaller size of scFvs provides better penetration into tumor tissue, improved pharmacokinetics, and a reduction in the immunogenicity observed with i.v. administered Fabs compared to that of intact murine antibodies (Bird et al., 1988; Cocher et al., 1990; Colcher et al., 1998; Adams and Schier, 1999). For example, the scFvMEL single-chain antibody retains the same binding affinity and specificity of the parental ZME-018 antibody that recognizes the surface domain of the gp240 antigen present on human melanoma cells (Kantor et al., 1982; Macey et al., 1988).

Recombinant single-chain Fv antibody (scFv)-based agents have been used in pre-clinical studies for cell-targeted delivery of cytokines (Liu et al., 2004) and intracellular delivery of highly cytotoxic n-glycosidases such as recombinant gelonin (rGel) toxin (Rosenblum et al., 2003). The smaller size of these antibody fragments may allow better penetration into tumor tissue, improved pharmacokinetics, and a reduction in the immunogenicity observed with intravenously administered murine antibodies. Initially, to target melanoma cells, we chose a recombinant single-chain antibody designated scFvMEL which recognizes the high-molecular-weight glycoprotein gp240, found on a majority (80%) of melanoma cell lines and fresh tumor samples (Kantor et al., 1982). It has been used extensively by the present inventors to target gp240 bearing cells in vitro and using xenograft models (Rosenblum et al., 2003; Liu et al., 2003; Rosenblum et al., 1991; Rosenblum et al., 1994; Rosenblum et al., 1995; Rosenblum et al., 1996; Rosenblum et al., 1999). This antibody binds to target cells and is efficiently internalized making this an excellent carrier to deliver t therapeutic payloads such as IkB.

Antibodies designated ZME-018 or 225.28 S that is the parental antibody of scFvMEL targeting the gp240 antigen have been extensively studied in melanoma patients and have demonstrated an impressive ability to localize in metastatic tumors after systemic administration (Rosenblum et al., 1994; Kantor et al., 1986; Macey et al., 1988; Rosenblum et al., 1991). This antibody possesses high specificity for melanoma and is minimally reactive with a variety of normal tissues, making it a promising candidate for further study (Rosenblum et al., 1995; Macey et al., 1988; Rosenblum et al., 1991; Mujoo et al., 1995). More importantly, the gp240 antigen is not expressed on normal cells thus making this an interesting target for therapeutic intervention.

The variable regions from the heavy and light chains (VH and VL) are both approximately 110 amino acids long. They can be linked by a 15 amino acid linker or longer with the sequence, for example, which has sufficient flexibility to allow the two domains to assemble a functional antigen binding pocket. In specific embodiments, addition of various signal sequences allows the scFv to be targeted to different organelles within the cell, or to be secreted. Addition of the light chain constant region (Ck) allows dimerization via disulfide bonds, giving increased stability and avidity. Thus, for a single chain Fv (scFv) SCA, although the two domains of the Fv fragment are coded for by separate genes, it has been proven possible to make a synthetic linker that enables them to be made as a single protein chain scFv (Bird et al., 1988; Huston et al., 1988) by recombinant methods. Furthermore, they are frequently used due to their ease of isolation from phage display libraries and their ability to recognize conserved antigens (for review, see Adams and Schier, 1999). For example, scFv is utilized to target suicide genes to carcinoembryonic antigen (CEA)-expressing tumor cells by a retrovector displaying anti-CEA scFv (Kuroki et al., 2000).

Recombinant single-chain Fv antibody (scFv)-based agents have been used in preclinical studies for cell-targeted delivery of cytokines and intracellular delivery of highly cytotoxic n-glycosidases such as recombinant gelonin (rGel) toxin (Liu et al., 2004; Lyu et al., 2005; Rosenblum et al., 1999; Rosenblum et al., 2003). The smaller size of these antibody fragments may allow better penetration into tumor tissues, improved pharmacokinetics, and reduction in the immunogenicity observed with intravenously administered murine antibodies (Colcher et al., 1990; Savage et al., 1993). For example, to target melanoma cells, a recombinant single-chain antibody scFvMEL may be used. This antibody retains the same binding affinity and specificity of the parental ZME-018 antibody recognizing the surface domain of the gp240 antigen. The scFvMEL antibody has been used extensively in to target gp240-bearing cells in vitro and using xenograft models (Liu et al., 2003, Liu et al., 2004; Rosenblum et al., 1991, Rosenblum et al., 1995; Rosenblum et al., 1996; Rosenblum et al., 2003). This antibody binds to target cells and is efficiently internalized, making it an excellent carrier for the delivery IkB.

6. Bispecific Antibodies

Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, 1983). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published May 13, 1993), and in Traunecker et al. (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in copending application Ser. No. 07/931,811 filed Aug. 17, 1992. For further details of generating bispecific antibodies see, for example, Suresh et al. (1986).

7. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

IV. Conjugation or Linkage of IkB and Cell Targeting Moieties

The cell targeting constructs of the invention may be joined by a variety of conjugations or linkages that have been previously described in the art. In one example, a biologically-releasable bond, such as a selectively-cleavable linker or amino acid sequence may be used. For instance, peptide linkers that include a cleavage site for an enzyme preferentially located or active within a tumor environment are contemplated. For example, linkers that are cleaved by urokinase, plasmin, thrombin, Factor IXa, Factor Xa, or a metallaproteinase, such as collagenase, gelatinase, or stromelysin. In a preferred embodiment, a linker that is cleaved by an intracellular proteinase is preferred, since this will allow the targeting construct to be internalized intact into targeted cells prior to cleavage.

Amino acids such as selectively-cleavable linkers, synthetic linkers, or other amino acid sequences such as the glycine rich linkers are described above and may be used to separate proteinaceous components. In some specific examples linkers for use in the current invention include the 218 linker (GSTSGSGKPGSGQGSTKG) (SEQ ID NO:1) or the G₄S linker (GGGGS) (SEQ ID NO:2). Additionally, while numerous types of disulfide-bond containing linkers are known that can successfully be employed to conjugate the IkB with a cell targeting moiety, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the toxin moiety prior to binding at the site of action.

Additionally, any other linking/coupling agents and/or mechanisms known to those of skill in the art can be used to combine the components of the present invention, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

It is contemplated that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxysuccinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Thorpe et al., 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

V. Therapeutic Regimens for Use in Conjunction with Cell Targeted IkB

As detailed above in certain embodiments of the invention, there is provided a method for sensitizing a cell to a cytotoxic therapy. It will be understood that cytotoxic therapies may include but are not limited to chemotherapy, immunotherapy, gene therapy and radiation therapy. It is envisioned that in any case cell targeting constructs according to the invention may be delivered before, after or with other cytotoxic therapies. However, in preferred embodiments the cell targeting moiety is delivered prior to or simultaneously with the cytotoxic therapy. Some examples of cytotoxic therapies for use with cell targeted IkB are indicated below.

1. Cytotoxic Therapies for Use with Cell Targeted IkB

Chemotherapy

In certain embodiments of the invention targeted IkB delivery is administered in conjunction with a chemo therapeutic agent. For example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, gemcitabien, navelbine, farnesyl-protein tansferase inhibitors, transplatinum, 5-fluorouracil, vincristin, Velcade, vinblastin and methotrexate, or any analog or derivative variant of the foregoing may used in methods according to the invention.

In certain cases it may also be preferable to administer an NF-kB inhibiting composition in conjunction with targeted IkB. Some compounds are known to inhibit NF-kB activity include but are not limited to curcuminoids, avicins, CAPE, capsaicin, sanguinarin, PTPase inhibitors, lapachone, resveratrol, vesnarinone, leflunomide, anethole, PI3 kinase inhibitors, oleanderins, emodin, serine preotease inhibitors, protein tyrosine kinase inhibitors, thalidomide, methotrexate. It has also recently been demonstrated that certain selinium compounds such as sodium seleite, methylseleninic acid and methylselenol inhibit NF-kB and exhibit anticancer activity (Gasparian et al., 2002).

Radiotherapy

In certain preferred embodiments of the invention cell targeted IkB may be used to sensitize cell to radiation therapy. Radio therapy may include, for example, □-rays, X-rays, and/or the directed delivery of radioisotopes to tumor cells. In certain instances microwaves and/or UV-irradiation may also used according to methods of the invention. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radio therapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B, Her-2/neu, gp240 and p155.

Genes

In yet another embodiment, gene therapy in which a therapeutic polynucleotide is administered before, after, or at the same time as a cell targeting construct of the present invention. Delivery of a cell targeted IkB in conjunction with a vector encoding one of the following gene products will have a combined anti-hyperproliferative effect on target tissues. A variety of genes are encompassed within the invention, for example a gene encoding p53 may be delivered in conjunction with IkB.

2. Secondary Treatments for Use with Cell Targeted IkB

Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. The cell targeted IkB of the present invention may be employed alone or in combination with a cytotoxic therapy as neoadjuvant surgical therapy, such as to reduce tumor size prior to resection, or it may be employed as postadjuvant surgical therapy, such as to sterilize a surgical bed following removal of part or all of a tumor.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

In the case wherein the targeted IkB is for the treatment of an autoimmune disease standard treatments for that disease may also be administered. For example steroidal drugs may be administered in conjunction with the targeted IkB.

3. Therapies for Autoimmune Diseases and Inflammatory Diseases.

Cell targeting constructs according to the instant invention may also be used in conjunction with other therapies that are used for the treatment of inflammation and/or autoimmune diseases. Thus, one may use a nucleic acid construct encoding an ATPase or ADPase activity and/or a polypeptide encoding the ATPase or ADPase activity in combination with an anti-inflammatory agent. Anti-inflammatory agents are agents that decrease the signs and symptoms of inflammation. A wide variety of anti-inflammatory agents are known to one of skill in the art. Most commonly used are the nonsteroidal anti-inflammatory agents (NSAIDs) which work by inhibiting the production of prostaglandins. Non-limiting examples include, ibuprofen, ketoprofen, piroxicam, naproxen, naproxen sodium, sulindac, aspirin, choline subsalicylate, diflunisal, oxaprozin, diclofenac sodium delayed release, diclofenac potassium immediate release, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate. Another group of anti-inflammatory agents comprise steroid based potent anti-inflammatory agents, for example, the corticosteroids which are exemplified by dexamethason, hydrocortisone, methylprednisolone, prednisone, and triamcinolone as non-limiting examples. Several of these anti-inflammatory agents are available under well known brand names, for example, the NSAIDs comprising ibuprofen include Advil, Motrin IB, Nuprin; NSAIDs comprising acetaminophens include Tylenol; NSAIDs comprising naproxen include Aleve.

VI. Administration of Cell Targeting Constructs

In some embodiments, an effective amount of a cell targeting constructs of the invention are administered to a cell. In other embodiments, a therapeutically effective amount of the targeting constructs of the invention are administered to an individual for the treatment of disease. The term “effective amount” as used herein is defined as the amount of the cell targeted IkB of the present invention that is necessary to result in a physiological change in the cell or tissue to which it is administered either when administered alone or in combination with a cytotoxic therapy. The term “therapeutically effective amount” as used herein is defined as the amount of the targeting molecule of the present invention that eliminate, decrease, delay, or minimize adverse effects of a disease, such as cancer. A skilled artisan readily recognizes that in many cases cell targeted IkB may not provide a cure but may only provide partial benefit, such as alleviation or improvement of at least one symptom. In some embodiments, a physiological change having some benefit is also considered therapeutically beneficial. Thus, in some embodiments, an amount of cell targeted IkB that provides a physiological change is considered an “effective amount” or a “therapeutically effective amount.” It will additionally be clear that a therapeutically effective amount may be dependent upon the inclusion of additional therapeutic regimens tat administered concurrently or sequentially. Thus it will be understood that in certain embodiments a physical change may constitute an enhanced effectiveness of a second therapeutic treatment.

The cell targeting constructs of the invention may be administered to a subject per se or in the form of a pharmaceutical composition for the treatment of cancer, autoimmunity, transplantation rejection, post-traumatic immune responses and infectious diseases, for example by targeting viral antigens, such as gp120 of HIV. More specifically, the chimeric polypeptides may be useful in eliminating cells involved in immune cell-mediated disorder, including lymphoma; autoimmunity, transplantation rejection, graft-versus-host disease, ischemia and stroke. Pharmaceutical compositions comprising the proteins of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations which can be used pharmaceutically. Proper formulation is dependent upon the route of administration chosen.

In a preferred embodiment of the invention cancer cells that may be treated by methods and compositions of the invention. Cancer cells that may be treated with cell targeting constructs according to the invention include but are not limited to cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In preferred embodiments systemic formulations of the cell targeting constructs are contemplated. Systemic formulations include those designed for administration by injection, e.g. subcutaneous, intravenous, intramuscular, intrathecal or intraperitoneal injection, as well as those designed for transdermal, transmucosal, inhalation, oral or pulmonary administration. In the most preferred embodiments cell targeted IkB is delivered by direct intravenous or intratumoral injection.

For injection, the proteins of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents.

Alternatively, the proteins may be in powder form for constitution with a suitable vehicle, e.g., sterile pyrogen-free water, before use.

Effective Dosages

The cell targeted IkB of the invention will generally be used in an amount effective to achieve the intended purpose. For use to treat or prevent a disease condition, the molecules of the invention, or pharmaceutical compositions thereof, are administered or applied in a therapeutically effective amount. A therapeutically effective amount is an amount effective to ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC5 as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the molecules which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation.

The amount of molecules administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of autoimmune disorders, the drugs that may be used in combination with IL2-Bax of the invention include, but are not limited to, steroid and non-steroid anti-inflammatory agents.

Toxicity

Preferably, a therapeutically effective dose of the cell targeted IkB described herein will provide therapeutic benefit without causing substantial toxicity.

Toxicity of the molecules described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. Proteins which exhibit high therapeutic indices are preferred. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975).

Pharmaceutical Preparations

Pharmaceutical compositions of the present invention comprise an effective amount of one or more chimeric polypeptides or chimeric polypeptides and at least one additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one chimeric polypeptide or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The cell targeted IkB may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where compositions according to the invention are provided in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Construction Antibody Fusion Proteins

As an example of IkB antibody fusion proteins, a chimeric fusion protein between human IkBα and the single chain antibody scFvMEL is described. The IkBα/scFvMEL fusion protein is composed of human IkBα fused to the single-chain antimelanoma antibody scFvMEL via a short, flexible tether (G₄S). For the construction of this fusion, the human IkBα gene was cloned from HL-60 cell RNA by reverse transcription polymerase chain reaction (RT-PCR) with the primers NTXIKB (5′ to 3′): CTGGTGCCACGCGGTTCTTTCCAGGCGGCCGAGCGC (SEQ ID NO:5) and CG4SIKB (5′ to 3′): GGAGCCACCGCCACCTAACGTCAGACGCTG (SEQ ID NO:6). These primers were designed to insert a thrombin cleavage site (SEQ ID NO:4) at the NH₂ (amino) terminus of IkB. Construction of the fusion protein was based on an overlapping PCR method. scFvMEL gene was amplified from plasmid pET32-scFvMEL/TNF previously described by PCR using the primers NG4SMEL (5′ to 3′): GGTGGCGGTGGCTCCACGGACATTGTGATG (SEQ ID NO:7) and CH3MEL (5′ to 3′): GCAGATGCTACCAAGCTTTCATTATGAGGAGACGGTGAG (SEQ ID NO:8). The IkBα and scFvMEL genes were linked together by using primers NTXIKB and CH3MEL.

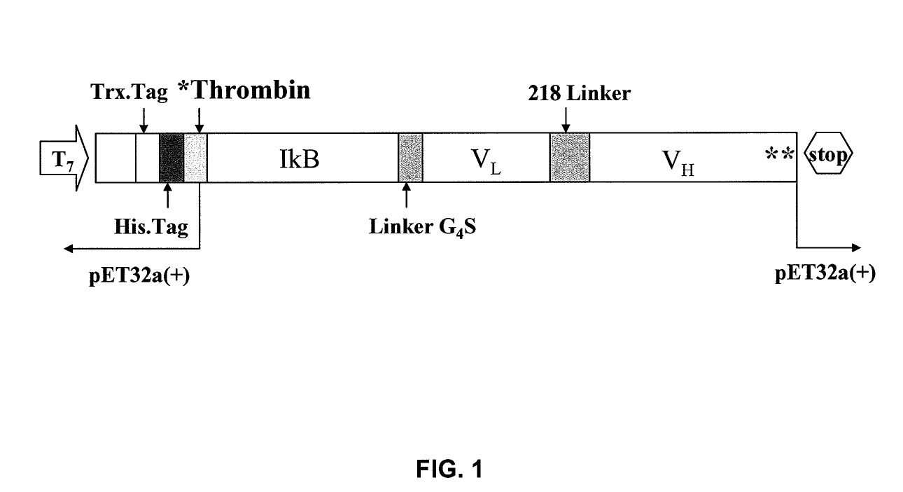

The fused genes with NH₂ terminal thrombin cleavage site were next subcloned into pET-32a (+) vector. The fragment from pET-32a (+) was amplified by using the primers T7 promoter (5′ to 3′): TAATACGACTCACTATAG (SEQ ID NO:9) and CPETTX (5′ to 3′): AGAACCGCGTGGCACCAGACCAGAAGAATG (SEQ ID NO:10). Next IkBα-scFvMEL fusion gene was cloned into the pET-32a (+) vector at XbaI and Hind III endonuclease sites. This construct was designated pET-32 IkBα/scFvMEL and is depicted in FIG. 1. As shown, the vector comprises a T7 promoter for high-level expression followed by a Trx.tag, a His.tag, a thrombin cleavage site, and a recombinant enterokinase (EK) cleavage site for final removal of the protein purification tag. In the construction described here, the EK cleavage site was deleted. Thus, fusion proteins synthesized from the construct may be cleaved with thrombin resulting in only two additional amino acids (GlySer) at the NH₂ terminus of processed fusion construct. These additional two amino acids were not detrimental to the biology activity of the fusion protein.

### Example 2

### Expression and Purification of Fusion Proteins

The recombinant protein IkBα/scFvMEL was transformed into Origami (DE3) E. coli for expression. Transformed bacteria were grown in Luria broth containing 400 μg/ml carbenicillin, 15 μg/ml kanamycin, and 15 μg/ml tetracycline, at 37° C. overnight in a shaking incubator at 240 rpm. The following day cultures were diluted 1:100 in fresh Luria broth plus antibiotics (200 μg/ml ampicillin, 15 μg/ml kanamycin, and 15 μg/ml tetracycline), grown at 37° C. until the absorbance at 600 nm was 1.0, and then diluted 1:1 with fresh medium with antibiotics. At this point expression of fusion protein was induced by addition of isopropyl β-D-thiogalacto-pyranoside (IPTG) to a final concentration of 100 μM and the bacteria were incubated overnight at 23° C. These cells were harvested, resuspended in 10 mM Tris-HCl (pH 8.0) and stored frozen at −20° C. for later purification.

In order to purify the fusion proteins from bacterial cells, the cells were resuspended by sonication. The supernatant was centrifuged at 186,000 g (Ti45 rotor) for 1 hr. The remaining soluble supernatant was adjusted to 20 mM Tris-HCl (pH 8.0), 500 mM NaCl and loaded onto a nickel-charged metal-affinity column preequilibrated with the same buffer. The column washed with buffer containing 20 mM imidazole and bound proteins were eluted with buffer containing 200 mM imidazole. Absorbance (280 nm) and sodium dodecyl-sulfate polyacrylamide gel electrophoresis (SDS-PAGE) analyses were performed to determine which fraction(s) contained the majority of the ˜77 kDa polyhistidine-tagged (His-tag) protein. Fractions were combined and dialyzed against 20 mM Tris-HCl (pH 8.0), 150 mM NaCl. The fusion protein was incubated with thrombin (1 unit of thrombin cleaves 5 mg protein when incubated at room temperature for 16 hours) to remove the His-tag. The mixture was further purified using cobalt-charged chelating sepharose resin to remove incompletely digested material and the cut-off his-tag. Analysis of the protein by SDS-PAGE and Coomassie stain demonstrated that the ˜63 kDa protein was substantially free of the of the cleaved ˜14 kDa His-tagged fragment. The final protein product was dialyzed in PBS and stored at 4° C. It was determined that approximately 2 mg of purified fusion protein could be obtained per liter of bacteria that were cultured.

### Example 3

### Internalization of IkBα/scFvMEL into gp240 Antigen Positive Human Melanoma Cells in Culture

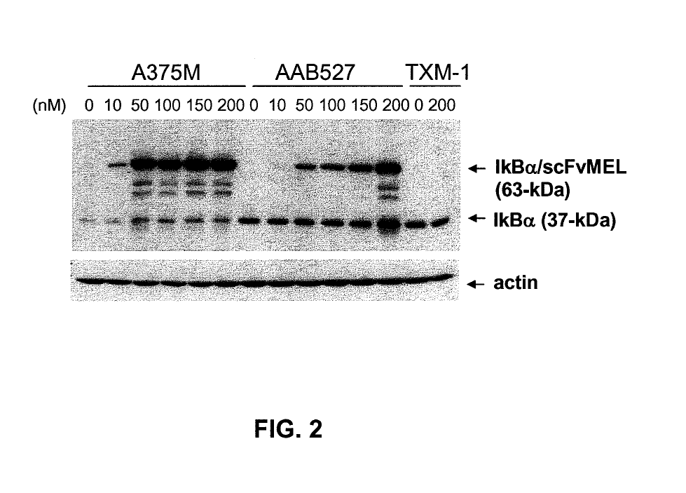

To examine the internalization of IkBα/scFvMEL, gp240 antigen positive A375-M and AAB-527 as well as gp240 antigen negative TXM-1 cells were treated with IkBα/scFvMEL at different concentrations for 2 hours. Following administration cell surfaces were washed and stripped by glycine buffer (0.5 M NaCl, 0.1 M glycine, pH 2.5) for 5 minutes to remove excess fusion protein. Cells were lysed and proteins were analyzed by Western blot and detected by rabbit anti-IkB antibody (via the protocol detailed in Example 7). The endogenous IkBα (37-kDa) could be detected in all cells by anti-IkB antibody. In gp240 antigen positive A375-M and AAB-527 cells treated with 200 nM IkBα/scFvMEL for 2 hours, a 63-kDa protein was detected with the anti IkB antibody that was not present in these cells treated with PBS (0 nM IkBα/scFMEL). The intensity of the 63-kDa bands was reduced in AAB-527 cells treated with 150 nM and was very low in AAB-527 cells treated with 50 nM IkBα/scFvMEL (FIG. 2). Therefore, intracellular IkBα/scFvMEL levels are directly proportional to its concentration in the culture medium in AAB-527 cells. However, the intensity of the 63-kDa band was very high and approximately similar in A375-M cells treated with 50-200 nM IkBα/scFvMEL (FIG. 2). These different patterns of internalization in these two gp240 antigen positive cells may be due to the different expression and/or distribution of the gp240 antigen on the cell surface. The 63-kDa IkBα/scFvMEL protein was not present in gp240 antigen negative TXM-1 cells incubated with 200 nM IkBα/scFvMEL, further demonstrating the cell specification of the targeting construct (FIG. 2). These data demonstrate that scFvMEL can effectively mediate delivery of IkBα to gp240 antigen positive cells.

Cell Culture

Human promyelocytic cell line HL-60 was obtained from American Type Culture Collection (ATCC, Manassas, Va.) and used to clone human IkBα gene. HL-60 cells were maintained in Iscove's modified Dulbecco's medium with 4 mM L-glutamine and 20% fetal bovine serum (FBS). Different human melanoma cell lines were used to study the radiosensitizing effect of inhibition of NF-kB: A375-M, A375SM (gp240 antigen positive) and TXM-1 (gp240 antigen negative). Human melanoma AAB527 (gp240 antigen positive) cells were also used to study internalization of the fusion protein. A375-M, A375SM, AAB-527, and TXM-1 cells were cultured in Dulbecco's MEM containing 10% FBS, with added sodium pyruvate (1 mM), non-essential amino acids (0.1 mM), L-glutamine (2 mM), and MEM vitamins. All cells were grown at a density of ˜7×10⁶ cells/T-75 flask, subcultured and were routinely tested and found to be free of mycoplasma contamination using the Mycoplasma Plus™ PCR Primer Sets (Stratagen, Cedar Creek, Tex.). Tissue culture media and supplements were purchased from Life Technologies Inc. (Rockville, Md.).

### Example 4

### Localization of the IkBα/scFvMEL Fusion Construct in Mice Bearing A375-M Xenograft Tumors

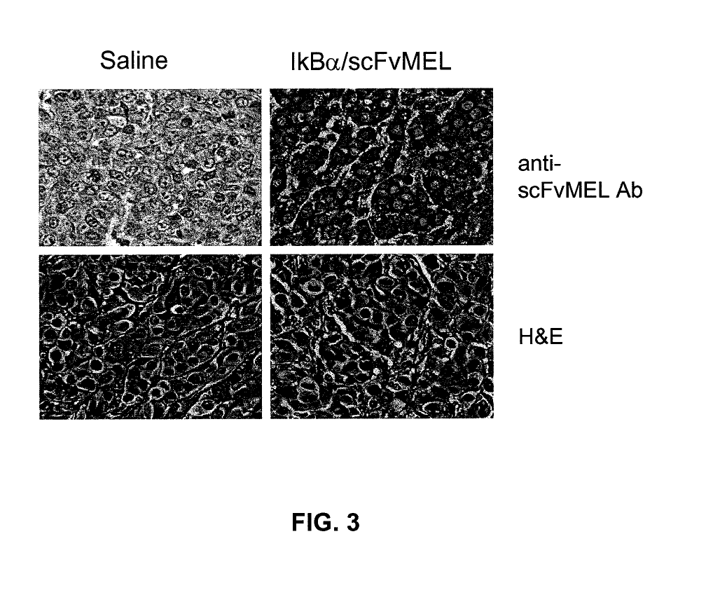

The ability of the IkBα/scFvMEL fusion protein to target gp240 positive tumors was initially analyzed in a murine xenograft tumor model. Athymic (nu/nu) mice, 4 to 6 weeks old, were obtained from Harlan Sprague Dawley (Indianapolis, Ind.). The animals were maintained under specific pathogen-free conditions and were used at 6 to 8 weeks of age. Animals were injected subcutaneously (right flank) with 3×106 log-phase A375-M melanoma cells, and tumors were allowed to establish. Once tumors had reached a measurable size (˜30-50 mm³), animals were treated through the intravenous tail vein with either saline (control) or IkBα/scFvMEL fusion construct daily for ten days with a total dose of 100 mg/kg. Twenty four hours after administration of IkBα/scFvMEL mice were sacrificed, and tumor tissues analyzed. In each case tumors were formalin-fixed, subjected to hematoxylin-and-eosin (H&E) staining, and scFvMEL was detected by an anti-scFvMEL antibody. Results for these studies clearly demonstrated that IkBα/scFvMEL localization to tumor tissue and was internalization into tumor cells (FIG. 3).

### Example 5

### The IkBα/scFvMEL Fusion Protein Blocks Constitutive and Radiation-Induced Activation of NF-kB

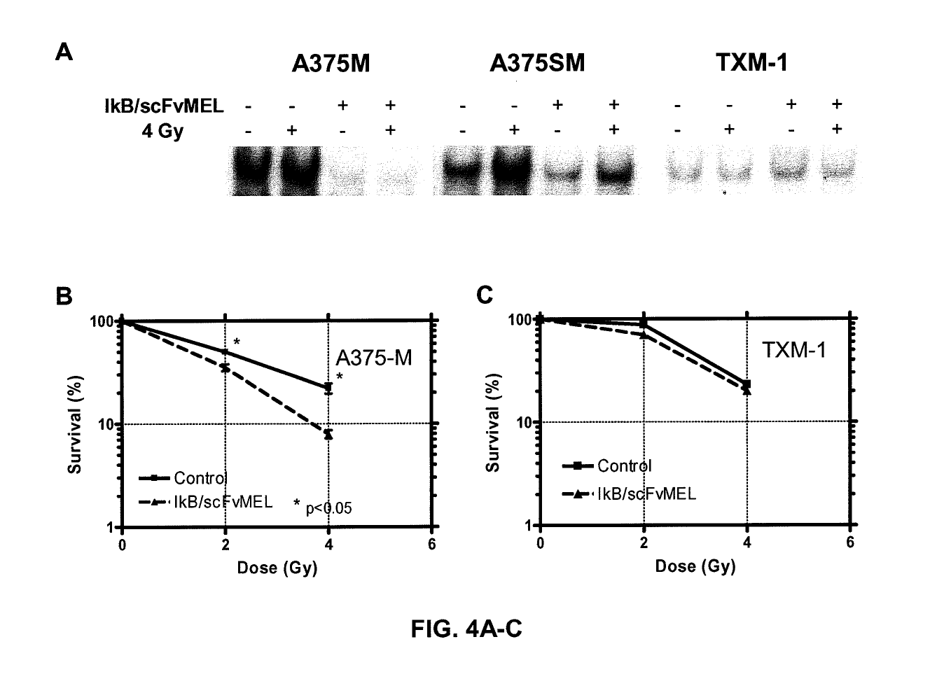

It has been shown previously that NF-kB is constitutively activated in tumors of different origins including melanomas (Huang et al., 2000; Yang and Richmond, 2001). Thus, the constitutive and radiation induced NF-kB activity in three human melanoma cell lines, A375-M, A375SM and TXM-1 was investigated and the effect of IkBα/scFvMEL on NF-kB activity determined. In each case cells were harvested after the treatments and nuclear extracts prepared as outline below. NF-kB activity was then assessed in each extract by electromobilty shift assay (EMSA) to detect amount of transcriptionally active NF-kB, see the protocol below. Studies represented in FIG. 4A demonstrate that NF-kB is constitutively activated in all three melanoma cell lines. Furthermore, induction of NF-kB in response to ionizing radiation was observed in A375-M and A375SM cells following a two hour exposure to a 4 Gy radiation dose (FIG. 4). However, administration of IkBα/scFvMEL fusion proteins was able to block constitutive activation of NF-kB in gp240 antigen positive A375-M and A375SM but not in gp240 antigen negative TXM-1 cells. In addition, induction of NF-kB activity by ionizing radiation in both gp240 antigen positive cell lines was blocked by a 2-hour pre-treatment with 0.3 μM IkBα/scFvMEL (FIG. 4). These studies demonstrate that internalized IkB fusion proteins can block both constitutive and induced NF-kB activation specifically in the targeted cell populations.

Nuclear Extract Preparation

Cells are rinsed twice with ice-cold PBS, harvested by scraping with a cell scraper, and centrifuged at 800 rpm for 10 minutes at 4° C. The cell pellet was resuspended in 400 μl of cold lysis buffer containing 10 mM HEPES (pH 7.9), 10 mM KCl, 0.1 mM EDTA, 0.1 mM EGTA, 1 mM DTT, 2 μg/ml leupeptin, 2 μg/ml aprotinin, and 0.5 mM phenylmethylsulfonyl fluoride. The mixture was incubated on ice for 10 minutes then 10% NP40 was added and the mixture was vortexed for 5 seconds. The lysate was centrifuged for 5 minutes at 4° C. (14,000 rpm). The supernatant was removed and stored as cytosolic extract. The pellet was resuspended in 30 μl of extraction buffer containing 20 mM HEPES (pH 7.9), 400 mM NaCl, 1 mM EDTA, 1 mM EGTA, 1 mM DTT, 2 μg/ml leupeptin, 2 μg/ml aprotinin, and 0.5 mM phenylmethylsulfonyl fluoride, mixed thoroughly, and incubated on ice for 30 minutes. The pellet was vortexed every 10 minutes. At the end of 30 minutes, the extract was centrifuged for 10 minutes at maximum speed (14,000 rpm) in a microcentrifuge. The supernatant was designated as nuclear extract, aliquoted, and stored at −70° C. until used in the EMSA.

Electrophoretic Mobility Shift Assay (EMSA)

Nuclear extracts from cells were run on EMSA to determine the extent of NF-kB activation in response to treatment with IkBα/scFvMEL and/or radiation. Essentially, nuclear extracts (15 μg) were incubated with poly(deoxyinosinic-deoxycytidylic acid) (1 μg) in binding buffer containing 10 mM Tris, 50 mM NaCl, 20% glycerol, 0.5 mM EDTA, and 1 mMDTT. [³²P]-labeled probe comprising an NF-kB binding site was added and allowed to bind for 15 minutes. The complexes were separated on a native 4% polyacrylamide gel and visualized by phosphorimaging.

### Example 6

### Treatment with IkBα/scFvMEL Fusion Construct Enhances Radiosensitivity in gp240 Antigen Positive Melanoma Cells in an In Vitro Clonogenic Survival Assay

To determine if inhibition of activated NF-kB can reverse the radio resistance of melanoma cells, gp240 antigen positive A375-M and gp240 antigen negative TXM-1 cells were pretreated with 0.3 μM IkBα/scFvMEL for 2 hours, and the cells were irradiated with the indicated dose of radiation and plated for clonogenic cell survival assay (see below). As shown in FIG. 4B, IkBα/scFvMEL treatment suppressed the clonogenic survival of A375-M cells in response to 2 Gy (p<0.05) of radiation from 50.2±1.06% in the control group to 35.4±2.75% in the fusion protein plus radiation treatment group. When radiation was administered at a higher dose of 4 Gy (p<0.05) the effects were even more dramatic with 19.8±2.50% in radiation alone control group surviving but only a 7.4±0.74% survival rate in the group co-treated with IkBα/scFvMEL. Conversely, no statistically significant sensitization was observed in gp240 antigen negative TXM-1 cells treated with irradiation compared with or without IkBα/scFvMEL treatment (FIG. 4C). Therefore, treatment with IkBα/scFvMEL sensitizes radiation resistant gp240 antigen positive A375-M cells to the cytotoxic effects of ionizing radiation. Furthermore, radiosensitization is dependent on the expression of the gp240 antigen as confirmed by the study demonstrating no effect of the IkBα/scFvMEL on gp240 negative cells.

Clonogenic Survival Assay

The effectiveness of the combination of IkBα/scFvMEL and ionizing radiation was assessed by clonogenic assay. Melanoma cells were either pre-treated with PBS or IkBα/scFvMEL (0.3 μM) for 2 hours. Then cells were irradiated with various doses of ionizing radiation and then processed for clonogenic cell survival assay (Munshi et al., 2005). Following treatment, cells were trypsinized and counted. Known cell concentrations were replated in triplicate and returned to the incubator to allow macroscopic colony development. Colonies were stained with crystal violet solution and counted after ˜14 days. The percent plating efficiency and the fraction of surviving cells following each treatment were calculated based on the survival of non-irradiated cells treated with the indicated agent.

### Example 7

### Effect of Inhibition of Activated NF-kB on it Antiapoptotic Transcriptional Targets In Vitro and In Vivo

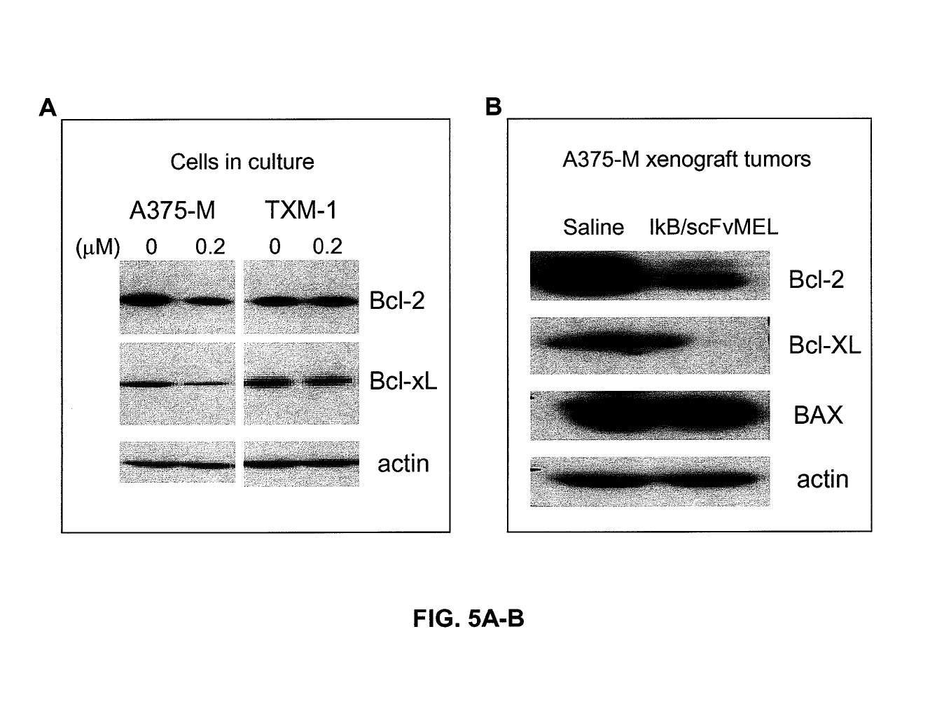

The effects of NF-kB inhibition on expression of downstream antiapoptotic targets such as Bcl-2 and Bcl-XL were determined by Western blot analysis. These studies demonstrated that treatment with IkBα/scFvMEL (0.2 μM for 2 hours) in culture decreased the levels of Bcl-2 and Bcl-XL in gp240 antigen positive A375-M cells but not in gp240 antigen negative TXM-1 cells (FIG. 5A). In addition, A375-M xenograft tumors in mice were also analyzed for expression of downstream NF-kB targets after treatment with IkBα/scFvMEL. Mice bearing A375-M xenograft tumors were administered IkBα/scFvMEL (100 mg/kg). Tumors were removed 24 hours after intravenous administration of IkBα/scFvMEL (100 mg/kg). Tumor tissue was homogenized in ice-cold lysis buffer containing protease inhibitors and centrifuged. Protein concentrations were then equalibrated between the samples and samples subjected to Western Blot analysis (see below) to determine Bcl-2 and Bcl-XL expression levels. Studies shown in FIG. 5B demonstrate that intravenous administration of IkBα/scFvMEL in mice bearing A375-M xenograft tumors caused a down-regulation of Bcl-2 and Bcl-XL proteins in tumors. Thus, IkBα/scFvMEL effectively down suppresses expression of antiapoptotic proteins downstream of NF-kB specifically in targeted cells.

Western Blotting Analyses

Antibodies to IkBα, Bcl2, Bcl-XL, Bax and actin were from Santa Cruz Biotechnology (Santa Cruz, Calif.). Cells were harvested after treatment, rinsed in ice-cold PBS, and lysed in lysis buffer containing 50 mM HEPES (pH 7.9), 0.4 M NaCl, 1 mM EDTA, 2 μg/ml leupeptin, 2 μg/ml aprotinin, 5 μg/ml benzamidine, 0.5 mM phenylmethylsulfonyl fluoride, and 1% Nonidet P-40 (NP40). The lysed cells were centrifuged at 14,000 rpm to remove cellular debris. Protein concentrations of the lysates were determined by the Bradford protein assay system (Bio-Rad, Hercules, Calif.). Equal amounts of protein were separated by 12% SDS-PAGE, transferred to polyvinylidene difluoride membranes (Millipore, Bedford, Mass.), and blocked with % nonfat milk in TBS-Tween 20 (0.05% v/v) for 1 hour at room temperature (RT). The membrane was incubated with the respective primary antibody for 1 hour at room temperature (RT). After washing, the membrane was incubated with the appropriate horseradish peroxidase (HRP) conjugated secondary antibody (Bio-Rad, Hercules, Calif.) for 1 hour. Following several washes, the blots were developed by enhanced chemiluminescence (Amersham Pharmacia Biotech, Arlington Heights, Ill.) and exposed to X-ray film.

Statistical Analyses

Data in each of the foregoing examples were analyzed using paired t test (Prism 3.0). Data are presented as mean±SE. A difference was regarded as significant if p<0.05.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,376,110
U.S. Pat. No. 4,554,101
U.S. Pat. No. 4,676,980
U.S. Pat. No. 4,680,338
U.S. Pat. No. 4,816,567
U.S. Pat. No. 5,141,648
U.S. Pat. No. 5,563,250
U.S. Pat. No. 5,821,337
U.S. Pat. No. 5,856,456
U.S. Pat. No. 5,880,270
U.S. Pat. No. 5,891,924
U.S. Pat. No. 6,214,974
U.S. Pat. No. 6,444,233
U.S. Pat. No. 7,083,957
U.S. Patent Appln. 2004005647
U.S. Patent Appln. 20060171919
U.S. Patent Appln. 20060234299
U.S. Patent Appln. 20060223114
U.S. patent application Ser. No. 07/715,272
U.S. patent application Ser. No. 07/931,811
Adams and Schier, J. Immunol. Methods, 231:249-260, 1999.
Aggarwal and Takada, Cancer Treat Res., 126:103-127, 2005.
Aggarwal et al., Indian J. Exp. Biol., 42:341-353, 2004.
Aggarwal, Cancer Cell, 6:203-208, 2004.
Agou et al., J. Biol. Chem., 279:54248-54257, 2004.
Atwell et al., Protein Eng., 12:597-604, 1999.
Baldwin Jr., Annu. Rev. Immunol., 14:649-683, 1996.
Barclay et al. (eds.), The Leucocyte Antigen Facts Book, 1993, Academic Press.
Barkett and Gilmore, Oncogene, 18:6910-6924, 1999.
Batra et al., Am. J. Respir. Cell Mol. Biol. 21:238-245, 1999.
Becker et al., J. Neuroimmunol., 77:27-38, 1997.
Bird et al., Science, 242:423-426, 1988.
Brodeur et al., In: Monoclonal antibody production techniques and applications, Marcel Dekker, Inc., NY, 51-63, 1987.
Clackson et al., Nature, 352:624-628, 1991.
Colcher et al., J. Natl. Cancer Inst., 82:1191-1197, 1990.
Colcher et al., J. Nucl. Med., 42:225-241, 1998.
David et al., Biochemistry, 13:1014, 1974.
Dhawan and Richmond, J. Biol. Chem., 277:7920-7928, 2002.
European Appln. 03089
Fingl et al., In: The Pharmacological Basis of Therapeutics, 1:1, 1975.
Foxwell et al., Ann. Rheum. Dis., 59:i54-i59, 2000.
Gasparian et al., Mol. Cancer Ther., 1:1079-1087, 2002
Ghosh et al., Annu. Rev. Immunol., 16:225-260, 1998.
Goding, In: Monoclonal Antibodies: Principles and Practice, 2d ed., Academic Press, Orlando, Fla., pp 60-61, 71-74, 1986.
Griffith et al., EMBO J., 12:725-734, 1993.
Huang et al., Clin. Cancer Res., 6:2573-2581, 2000.
Hunter et al., Nature, 144:945, 1962.
Huston et al., Biochemistry, 27(25):8945-8952, 1988.
Iimuro et al., J. Clin. Invest., 101:802-811, 1998.
Jakobovits et al., Nature, 362:255-258, 1993.
Jakobovits et al., Proc. Natl. Acad. Sci. USA, 90:2551-255, 1993.
Johnson et al., Curr. Opinion Struct. Biol., 3:564-571, 1993.
Jones et al., Nature, 321:522-525, 1986.
Kantor et al., Cancer Res., 46:5223-5228, 1986.
Kantor et al., Hybridoma., 1:473-482, 1982.
Karin, Oncogene, 18:6867-6874, 1999.
Kohler and Milstein, Nature, 256:495-497, 1975.
Kozbor, J. Immunol., 133(6):3001-3005, 1984.
Kuroki et al., Anticancer Res., 20(6A):4067-4071 2000.
Kyte and Doolittle, J. Mol. Biol., 157(1):105-132, 1982.
Liu et al., Int. J. Cancer, 108:549-557, 2004.
Liu et al., Mol. Cancer Ther., 2:1341-1350, 2003.
Lyu and Rosenblum, Mol. Cancer Ther., 4:1205-1213, 2005.
Macey et al., Am. J. Physiol. Imaging, 3:1-6, 1988.
Marks et al., Bio/Technol., 10:779-783, 1992.
Marks et al., J. Mol. Biol., 222:581-597, 1991.
McCafferty et al., Nature, 348:552-553, 1990.
Millstein and Cuello, Nature, 305:537-539, 1983.
Morrison et al., Proc. Nat. Acad. Sci USA. 81:6851, 1984.
Mujoo et al., Cancer Immunol. Immunother., 40:339-345, 1995.
Munshi et al., Methods Mol. Med., 110:21-28, 2005.
Munson and Pollard, Anal. Biochem., 107:220, 1980.
Nechushtan et al., J. Biol. Chem., 272:11597-603, 1997.
NCBI accession No. AAA79696
NCBI accession No. NP_{—}035038
NCBI accession No. XP_{—}343066
NCBI accession No. NP_{—}110494
NCBI accession No. CAA84619
Nygren, Histochem. and Cytochem., 30:407, 1982.
Onda et al., Cancer Res., 64:1419-1424, 2004.
Pain et al., J. Immunol. Meth., 40:219, 1981.
Pajonk et al., J. Natl. Cancer Inst., 91:1956-1960, 1999.
PCT Appln. WO 91/00360
PCT Appln. WO 92/200373
PCT Appln. WO 93/06213
PCT Appln. WO 93/08829
PCT Appln. WO 97/19179
PCT Appln. WO 2005/017188
PCT Appln. WO 2005/021722
Remington's Pharmaceutical Sciences, 18th Ed. Mack Printing Company, pp. 1289-1329, 1990.
Riechmann et al., Nature, 332:323-327, 1988.
Rosenblum et al., Cancer Commun., 3:21-27, 1991.
Rosenblum et al., Cancer Immunol. Immunother., 39:397-400, 1994.
Rosenblum et al., Cancer Immunol. Immunother., 40:322-328, 1995.
Rosenblum et al., Cancer Immunol. Immunother., 42:115-121, 1996.
Rosenblum et al., Cancer Res., 63:3995-4002, 2003.
Rosenblum et al., Clin. Cancer Res., 5:865-874, 1999.
Rosenblum et al., Mol. Biother., 3:6-13, 1991.
Savage et al., Br. J. Cancer, 68:738-742, 1993.
Suresh et al., Methods in Enzymology, 121:210-228, 1986.
Thompson (ed.) 1994, The Cytokine Handbook, Academic Press, San Diego.
Thorpe et al., J. Natl. Cancer Inst., 79(5):1101-1112, 1987.
Traenckner et al., EMBO J., 14:2876-2883, 1995.
Traunecker et al., EMBO, 10:3655-3659, 1991.
Verhoeyen et al., Science, 239:1534-1536, 1988.
von Minckwitz et al., Breast Cancer Res., 7:R616-626, 2005.
Waterhouse et al., Nucl. Acids Res., 21:2265-2266, 1993.
Winthrop et al., Clin. Cancer Res., 9:3845s-3853s, 2003.
Yang and Richmond, Cancer Res., 61:4901-4909, 2001.
Zola, In: Monoclonal Antibodies: A Manual of Techniques, CRC Press, Inc., pp. 147-158, 1987.

What is claimed is:

1. A cell targeting construct comprising a polypeptide inhibitor of NF-kB (IkB) conjugated to cell targeting moiety.

2. The cell targeting construct of claim 1, wherein polypeptide inhibitor of NF-kB is human IkBα.

3. The cell targeting construct of claim 1, wherein polypeptide inhibitor of NF-kB is human IkBαM.

4. The cell targeting construct of claim 1, wherein the cell targeting moiety is further defined as an immune cell targeting moiety.

5. The cell targeting construct of claim 1, wherein the cell targeting moiety is further defined as an infected cell targeting moiety.

6. The cell targeting construct of claim 5, wherein the infected cell is bacteria or virus infected cell.

7. The cell targeting construct of claim 6, wherein the cell targeting moiety binds to a bacterial encoded antigen.

8. The cell targeting construct of claim 6, wherein the infected cell is a virus infected cell.

9. The cell targeting construct of claim 8, wherein the cell targeting moiety binds to a virus encoded antigen.

10. The cell targeting construct of claim 1, wherein the cell targeting moiety is further defined as a cancer cell targeting moiety.

11. The cell targeting construct of claim 10, wherein the cancer cell is a lung, breast, brain, prostate, spleen, pancreatic, cervical, ovarian, head and neck, esophageal, liver, skin, kidney, leukemia, bone, testicular, colon or bladder cancer cell.

12. The cell targeting construct of claim 10, wherein the cell targeting moiety binds to a cancer cell antigen.

13. The cell targeting construct of claim 12, wherein cancer cell antigen is gp240.

14. The cell targeting construct of claim 1, wherein the cell targeting moiety is further defined as an antibody, a growth factor, a hormone, a peptide, an aptamer, or a cytokine.

15. The cell targeting construct of claim 14, wherein the antibody is further defined as a full-length antibody, chimeric antibody, Fab′, Fab, F(ab′)2, single domain antibody (DAB), Fv, single chain Fv (scFv), minibody, diabody, triabody, or a mixture thereof.

16. The cell targeting construct of claim 15, wherein the antibody is a scFv.

17. The cell targeting construct of claim 14, wherein the antibody is an anti-HER-2/neu antibody.

18. The cell targeting construct of claim 17, wherein the HER-2/neu antibody is scFv23.

19. The cell targeting construct of claim 14, wherein the antibody is an anti-gp240 antigen antibody.

20. The cell targeting construct of claim 19, wherein the anti-gp240 antigen antibody is scFvMEL.

21. The cell targeting construct of claim 14, wherein the cancer cell-targeting moiety comprises one or more growth factors.

22. The cell targeting construct of claim 21, wherein the growth factor is transforming growth factor, epidermal growth factor, insulin-like growth factor, fibroblast growth factor, BLyS, heregulin, platelet-derived growth factor, vascular endothelial growth factor (VEGF), or hypoxia inducible factor.

23. The cell targeting construct of claim 22, wherein the growth factor is VEGF.

24. The cell targeting construct of claim 14, wherein the cancer cell-targeting moiety comprises one or more hormones.

25. The cell targeting construct of claim 24, wherein the hormone is human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY3-36, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36.

26. The cell targeting construct of claim 14, wherein the cancer cell-targeting moiety comprises one or more cytokines.

27. The cell targeting construct of claim 14, wherein the cytokine is IL1, IL2, IL3, IL4, IL5, IL6, IL7, IL8, IL9, IL10, IL11, IL12, IL13, IL14, IL15, IL-16, IL-17, IL-18, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, IFN-γ, IFN-α, IFN-β, LT-β, CD40 ligand, Fas ligand, CD27 ligand, CD30 ligand, 4-1BBL, TGF-β, IL 1α, IL-1 β, IL-1 RA, MIF, IGIF, or a mixture thereof.

28. The cell targeting construct of claim 1, wherein the polypeptide NF-kB inhibitor is chemically conjugated to the cell targeting moiety.

29. The cell targeting construct of claim 1, wherein the polypeptide NF-kB inhibitor is conjugated to the cell targeting moiety though a covalent bond.

30. The cell targeting construct of claim 29, wherein the polypeptide NF-kB inhibitor and the cell targeting moiety is a fusion protein.

31. The cell targeting construct of claim 30, wherein the fusion protein is IkBα/scFvMEL.

32. The cell targeting construct of claim 30, wherein the polypeptide NF-kB inhibitor and the cell targeting moiety are separated by a linker region.

33. The cell targeting construct of claim 32, wherein the linker region a G₄S linker or a 218 linker.

34. The cell targeting construct of claim 1, wherein the polypeptide NF-kB inhibitor is conjugated to the cell targeting moiety though a non-covalent interaction.

35. The cell targeting construct of claim 34, wherein the polypeptide NF-kB inhibitor is conjugated to the cell targeting moiety through a biotin-avadin interaction.

36. A method of treating a patient with a cell proliferative disease comprising administering to the patient a cell targeting construct according to claim 1 in amount that is effective for the treatment of said disease.

37. The method of claim 36, wherein the cell proliferative disease is a cancer or precancerous condition.

38. The method of claim 37, wherein the cell proliferative disease is a cancer.

39. The method of claim 38, wherein the cancer is lung, breast, brain, prostate, spleen, pancreatic, cervical, ovarian, head and neck, esophageal, liver, skin, kidney, leukemia, bone, testicular, colon, or bladder cancer.

40. The method of claim 39, wherein the cancer is a skin cancer.

41. The method of claim 40, wherein the cancer is a melanoma.

42. The method of claim 36, further comprising administering a cytotoxic therapy.

43. A method of sensitizing a cell to cytotoxic therapy comprising administering to the cell an effective amount of cell targeting contruct according to claim 1.

44. The method of claim 42, wherein the cytotoxic therapy is chemotherapy, radiation therapy, gene therapy or immunotherapy.

45. The method of claim 44, wherein the cytotoxic therapy is radiation therapy.

46. The method of claim 44, wherein the cytotoxic therapy is a chemotherapy.

47. The method of claim 46, wherein the cytotoxic therapy is a chemotherapy comprises an agent that reduces NF-kB activity.

48. The method of claim 47, wherein the agent that reduces NF-kB activity is a curcuminoid, an avicin, CAPE, capsaicin, sanguinarin, a PTPase inhibitor, lapachone, resveratrol, vesnarinone, leflunomide, anethole, a PI3 kinase inhibitor, oleanderin, emodin, a serine preotease inhibitor, a protein tyrosine kinase inhibitor, thalidomide or methotrexate.

49. The method of claim 46, wherein the chemotherapy comprises paclitaxel, gemcitabin, 5-fluorouracil, etoposide, cisplatin, capothecin, vincristine, Velcade or doxorubicin.

50. The method of claim 37, wherein the cell proliferative disease is an autoimmune disease.

51. The method of claim 36, wherein the cell targeting construct induces apoptosis in target cells.

52. A method of treating a bacterial or viral infection comprising administering an effective amount a cell targeting composition according to claim 1.

53. A nucleic acid sequence encoding the cell targeting construct of claim 30.

54. A cell comprising the nucleic acid sequence of claim 53.
